(19) United States
(12) Patent Application Publication
(10) Pub. No.: US 2016/0278711 A1
(43) Pub. Date: Sep. 29, 2016

(54) ADAPTIVE REMOVAL OF THE CARDIAC ARTIFACT IN RESPIRATION WAVEFORM

(71) Applicant: Draeger Medical Systems, Inc., Andover, MA (US)

(72) Inventor: Georgios MALLAS, Los Angeles, CA (US)

(21) Appl. No.: 15/074,931

(22) Filed: Mar. 18, 2016

Related U.S. Application Data
(60) Provisional application No. 62/137,430, filed on Mar. 24, 2015.

(51) Int. Cl. A61B 5/00; A61B 5/0408; A61B 5/046; A61B 5/0205

(52) U.S. Cl. CPC A61B 5/7203; A61B 5/0205; A61B 5/0408; A61B 5/046; A61B 5/742; A61B 5/0245

(57) ABSTRACT

Cardiac artifacts can be removed from respiration waveforms by receiving a stream of respiration samples of a sensed respiration signal that collectively characterize respiration data for a patient. In addition, heart rate data is received that specifies a heart rate for the patient that is measured concurrently with the sensed respiration signal. Each respiration sample in the stream is continuously and adaptively filtered to result in a corresponding filtered respiration signal that removes cardiac artifacts. This filtering subtracts an earlier respiration sample having a delay equal to a period corresponding to the heart rate of the patient from the then current respiration sample. The filtered respiration signals can then be promoted. Related apparatus, systems, techniques, and articles are also described.

### RELATED APPLICATION

The current application claims priority to U.S. Pat. App. Ser. No. 62/137,430 filed on Mar. 24, 2015, the contents of which are hereby fully incorporated by reference.

### TECHNICAL FIELD

The subject matter described herein relates to adaptive removal of the cardiac artifact in respiration waveforms using an adaptive filter.

### BACKGROUND

Monitoring of patient vital signs is a standard procedure in the hospital in intensive care units (ICUs), the operating room (OR) and others. Respiration monitoring is extremely important in the neonatal ICU (NICU) due to the sudden infant death syndrome (SIDS), where an infant experiences a lethal apnea event. Respiration is typically monitored using impedance respiration (IR), which monitors a patient's respiration indirectly using the ECG electrodes, thus allowing monitoring of the respiration activity without requiring additional sensors. In particular, IR injects a high frequency modulated current across ECG Lead I (typical in the NICU) or Lead II (typical in adult monitoring) used to measure a patient's chest impedance. Breathing causes slight variations in a patient's chest impedance, which modulate the injected current and thus allow the patient monitor to reconstruct a respiration waveform. A healthy adult's respiration rate (RR) is typically between 12-20 breaths per minute (brpm), while a healthy neonate's RR is typically between 30-50 brpm. A typical noise-free and artifact-free IR waveform is sinusoidal in nature, and of amplitudes 0.5-1.5 Ohm peak-to-peak.

However, IR is frequently corrupted by artifacts caused either by motion or by the function of the heart. The latter artifact type, which will be referred to herein as the cardiac artifact, is the result of impedance variations induced across the chest by circulating blood. Thus, the rate of the cardiac artifact coincides with that of the heart rate, while its amplitude is typically between 0.01-0.5 Ohms, which, as expected, is additive to changes in impedance induced by breathing. Note that typical heart rates for healthy adults and neonates are in the range of 55-105 beats per minute (bpm) and 120-160 bpm respectively.

Even though patient monitoring was introduced in the hospitals more than 30 years ago, there have been renewed efforts towards the improvement of existing monitoring technologies. The main driving factor for improvement is the fact that more than 80% of patient monitor alarms are false positives. This extremely high false alarm rate is due to the fact that, in order to achieve high sensitivity for life-threatening conditions, patient monitors tend to sacrifice specificity. However, this has led physicians and nurses to become desensitized to monitor alarms (“alarm fatigue”), which in turn increases treatment errors. Data collected from hospitals using patient monitors have suggested that the primary source of false positive alarms in IR is the cardiac artifact. For example, the cardiac artifact is frequently responsible for false high respiration rates and it can also lead to missed apnea events (false negative), because it is present even during absence of breathing and can thus be mistakenly detected as breathing from the patient monitor. Note that these issues are especially prevalent in neonates, because of their large heart/body ratio, the fact that they exhibit shallow breathing, and in addition because they are monitored using Lead I (for reasons related to the ECG), which is much more prone to cardiac artifacts due to its location on the body.

### SUMMARY

Patient monitors are used on a daily basis to monitor patient vital signs in thousands of hospitals worldwide. One of the important parameters monitored, especially in neonates, is the respiration rate. A purpose of respiration monitoring is the detection of apneas (prolonged absence of breathing that can lead to patient death) and of critically high respiration rates. Patient monitors typically measure respiration activity using impedance respiration, which extracts a respiration signal indirectly by measuring the change in impedance caused by breathing across the chest or abdomen of a patient. However, cardiac activity can also introduce a measurable change in chest impedance. This change can appear as a periodic artifact in impedance respiration, and it often causes patient monitors to falsely detect high respiration rates, thus triggering false alarms and leading to alarm fatigue. In addition, the cardiac artifact persists during apneas, and can potentially cause apnea events to be missed, which could lead to a patient death. The current subject matter includes an adaptive filter, which uses ECG information in order to remove the cardiac artifact from the measured signal and to prevent such false detections.

In a first aspect, cardiac artifacts are removed from respiration waveforms by receiving a stream of respiration samples of a sensed respiration signal that collectively characterize respiration data for a patient. In addition, heart rate data is received that specifies a heart rate for the patient that is measured concurrently with the sensed respiration signal. Each current respiration sample in the stream is continuously adaptively filtered to result in a corresponding filtered respiration signal that removes cardiac artifacts. The filtering subtracts an earlier respiration sample having a delay equal to a period corresponding to the heart rate of the patient from the then current respiration sample. The filtered respiration signals can then be promoted.

The promoting can include, for example, at least one of: displaying the filtered respiration signals in an electronic visual display, persisting the filtered respiration signals in a physical data storage device, transmitting the filtered respiration signals over a wired or wireless computing network to a remote computing device, and loading the filtered respiration signals into memory of a computing device.

The respiration signal can comprise an impedance respiration waveform.

The heart rate data can be derived from an electrocardiogram (ECG) electrode set affixed to the patient. With such variations, the period can be extracted from the heart rate data.

In the case of heart rate data derived from an ECG electrode set, the adaptive filtering can include weighting the earlier respiration samples by a normalizing factor that is based on a value of a corresponding R-wave read from the heart data at a time matching the corresponding current respiration sample. The normalization factor can be equal to an amplitude of the corresponding R-wave normalized by a maximum R-wave amplitude over a respiration rate period.

Power of the cardiac artifact can be estimated by integrating a power density of the respiration sensed respiration signal across a frequency region centered on the heart rate. In addition, power of the respiration rate can be estimated by integrating a power density across a largest peak of a spectrum corresponding to a respiration rate. A signal power to cardiac artifact power ratio (SCR) can be computed by dividing the estimated power of the respiration rate by the estimated power of the cardiac artifact. The adaptive filtering can be activated when the SCR is below a pre-defined threshold. The adaptive filtering can be deactivated when the SCR is above a pre-defined threshold.

The adaptive filtering can be deactivated when the heart rate data indicates a ventricular arrhythmia.

Each of the receiving, receiving, filtering, and promoting can be implemented by at least one programmable data processor forming part of at least one computing device (e.g., a patient monitor, etc.).

In some variations, the operations can be implemented as part of a system including an electrocardiogram (ECG) circuit, and electrodes configured to be coupled to the ECG circuit and for affixation to the patient. In such variations, the electrodes and the ECG circuit in combination generate the sensed respiration signal.

In an interrelated aspect, a method for removing cardiac artifacts from respiration waveforms includes receiving a stream of respiration samples of a sensed respiration signal that collectively characterize respiration data for a patient. In addition, heart rate data is received that specifies a heart rate for the patient that is measured concurrently with the sensed respiration signal. Each current respiration sample in the stream is adaptively filtered to result in a corresponding filtered respiration signal that removes cardiac artifacts. The filtering subtracts a weighted sum of a plurality of earlier respiration samples (as opposed to a single sample) from the then current respiration sample. The filtered respiration signals can then be promoted.

In some variations, each of the plurality of earlier respiration samples can have a delay equal to an integer multiple of a period corresponding to the heart rate of the patient.

Non-transitory computer program products (i.e., physically embodied computer program products) are also described that store instructions, which when executed by one or more data processors of one or more computing systems, causes at least one data processor to perform operations herein. Similarly, computer systems are also described that may include one or more data processors and memory coupled to the one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including but not limited to a connection over a network (e.g. the Internet, a wireless wide area network, a local area network, a wide area network, a wired network, or the like), via a direct connection between one or more of the multiple computing systems, etc.

The subject matter described herein provides many technical advantages. For example, the current subject matter can remove/filter cardiac artifacts from respiration waveforms in a manner that is more effective and less computationally expensive as compared to conventional low-pass or notch filters.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is a diagram illustrating a patient having an ECG electrode set affixed thereto that is connected to a patient monitor;

FIG. 2 is a process flow diagram illustrating adaptive removal of cardiac artifacts from an impedance respiration waveform;

FIG. 3 is a series of plots illustrating: impedance respiration monitoring with clean and corrupted impedance respiration waveforms;

FIG. 4 is a series of plots illustrating example simulated respiration signal, simulated cardiac artifact, the corrupted respiration signal, and the filtered signal.

FIG. 5 is a series of plots illustrating the power spectrum of the simulated true respiration signal, corrupted signal, and filtered signal of FIG. 4;

FIG. 6 is a series of plots illustrating ECG signal input to an example adaptive filter, respiration signal before adaptive filtering, and respiration signal after filtering;

FIG. 7 is a series of plots illustrating the power spectrum of the raw respiration signal and the filtered respiration signal; and

FIG. 8 is a diagram illustrating an example of quantitation errors that can arise during filtering.

### DETAILED DESCRIPTION

The current subject matter is directed to the removal or other filtering of cardiac artifacts within respiration waveforms. While the current subject matter is described in connection with impedance respiration measurement in which changes in impedance of electrodes affixed to a patient are measured and correlated to breath, it will be appreciated that the techniques utilized herein can also be applied to other sources of breath/respiration data such as various contact and non-contact methods in which breath related data/respiration waveforms are generated. Stated differently, the current subject matter can be applied to respiration waveforms using impedance as well as using other techniques that do not involve impedance. The contact methods can include, for example, acoustic-based techniques, airflow-based techniques, other chest/abdominal movement detection techniques, transcutaneous CO₂ monitoring, blood oxygen saturation measurements. The non-contact methods including: radar-based respiration rate monitoring, optical-based respiration rate monitoring, and thermal sensor/imaging based respiration rate monitoring.

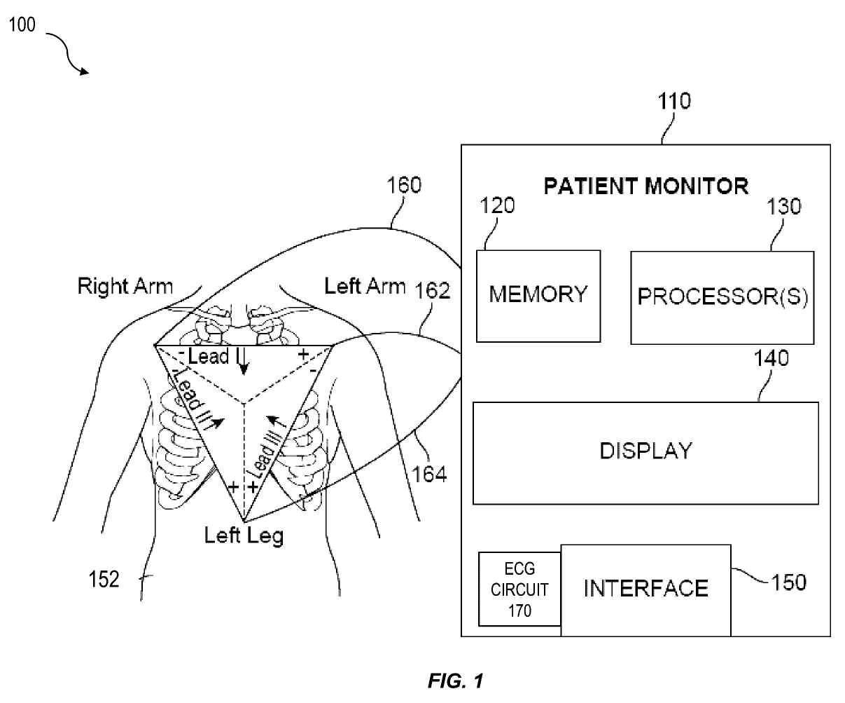

FIG. 1 is a diagram 100 illustrating an example implementation in which respiration rate of a patient 152 (represented by his or her torso) is measured by a patient monitor 110. The patient monitor 110 can include memory 120 for storing instructions for execution by one or more processor/processor cores 130. The patient monitor 110 can include a display 140 for rendering visual information that corresponds to the breathing rate (e.g., values, waveforms, etc.) as calculated using the techniques described herein by the processor(s) 130. In addition, the patient monitor 110 can also include an interface 150 that permits for wired or wireless communication with one or more electrodes 160, 162, and 164 and/or a remote medical device and/or a remote computing system or network to transmit/receive data pertaining to the rate of breath and the like. The patient monitor 110 can implement the processing described herein and, in other variations, the patient monitor 110 can transmit data characterizing the breaths of the patient 152 to a remote computing system (e.g., medical device, back-end computing system, etc.) via the interface 150 for a remote calculation of breath. An example patient monitor can include one described in U.S. Pat. No. 5,375,604 entitled “Transportable Modular Patient Monitor” and incorporated by reference herein in its entirety.

The interface 150 can include or otherwise be coupled to an ECG circuit 170 that directly or indirectly receives the outputs of the electrodes 160, 162, 164. The ECG circuit 170 can include at least one amplifier (e.g., an instrumentation amplifier, etc.) to amplify the signals received from the electrodes 160, 162, 164 as well as various filtering components/sub-circuits and, in some variations, a right leg drive circuit which helps reduce interference from the at least one amplifier. Other variations of the ECG circuit 170 can also be implemented.

The electrodes 160, 162, and 164 can form part of an electrocardiogram (ECG) electrode set in which electrode 160 is affixed to the right arm of the patient 152, electrode 162 is affixed to the left arm of the patient 152, and electrode 164 is affixed to the left leg of the patient 152. The positions of the electrodes 160, 162, and 164 form leads I, II, and III which, in turn, form points of what is referred to as Einthoven's triangle. Lead I is the voltage between the positive left arm electrode and the right arm electrode. Lead II is the voltage between the positive left leg electrode and the right arm electrode. Lead III is the voltage between the positive left leg electrode and the left arm electrode.

The impedance respiration monitoring techniques utilized herein can measure the change in impedance across the measured lead (e.g., lead I using electrodes 160, 162, lead II using electrodes 160, 164, etc.) and provide data/generate signals that characterize the breathing patterns of the patient 152. One or more of the electrodes 160, 162, and 164 can generate an output sometimes referred to as a sensed respiration signal that characterizes the breathing patterns of the patient. The respiration signal can, for example, characterize breathing patterns having periodic oscillations that correspond to breaths with peaks having amplitudes measured from a rate of equilibrium in between breaths. In particular, the patient monitor 110 can receive or otherwise calculate a sensed respiration signal (based on the physiological measurements of the patient 152) based on a stream of samples that are continuously received via, for example, the electrodes 160, 162, and 164. As will be described further below, such sensed respiration signal can include cardiac artifacts that can be filtered. In addition, with respiration monitoring, for a breath to be detected, the respiration signal must exceed a preset minimum amplitude threshold, which is typically between 0.15 and 0.2 Ohm peak to peak.

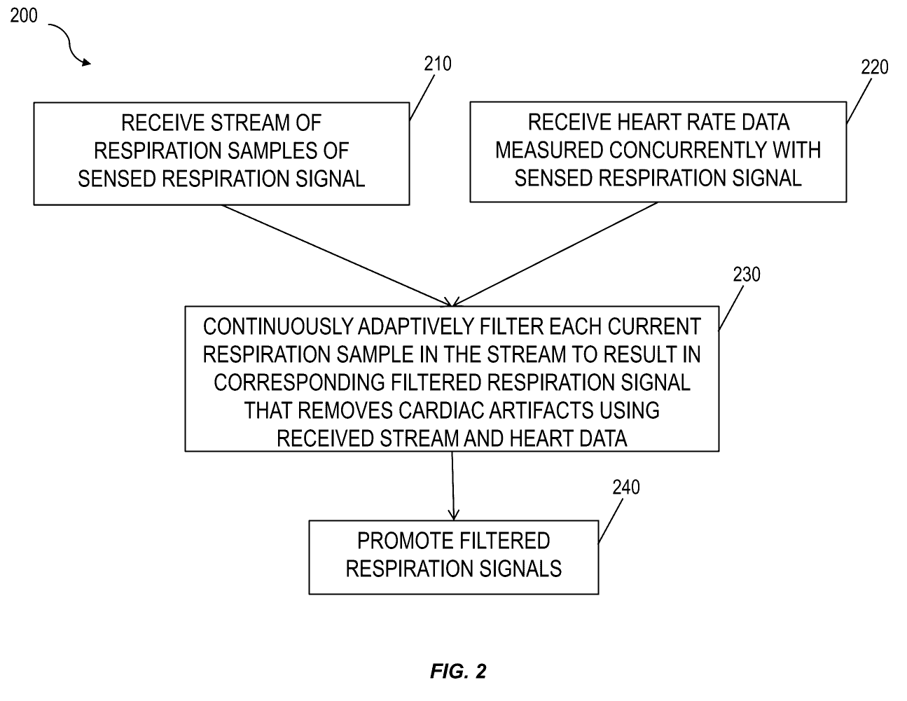

FIG. 2 is a process flow diagram 200 for removing cardiac artifacts from respiration waveforms. A stream of respiration samples of a sensed respiration signal is received, at 210, that collectively characterize respiration data for a patient. In addition, at 220, heart rate data is received that specifies a heart rate for the patient that is measured concurrently with the sensed respiration signal. The respiration samples in the stream are, at 230, continuously and adaptively filtered to each result in a corresponding filtered respiration signal that removes cardiac artifacts. The filtering subtracts an earlier respiration sample having a delay equal to a period corresponding to the heart rate of the patient from the then current respiration sample. The filtered respiration signals can then, at 240, be promoted. Promoting, in this regard, can include one or more of: displaying the filtered respiration signals in an electronic visual display, persisting the filtered respiration signals in a physical data storage device, transmitting the filtered respiration signals over a wired or wireless computing network to a remote computing device, or loading the filtered respiration signals into memory of a computing device. Further details of the cardiac artifact filtering are described below.

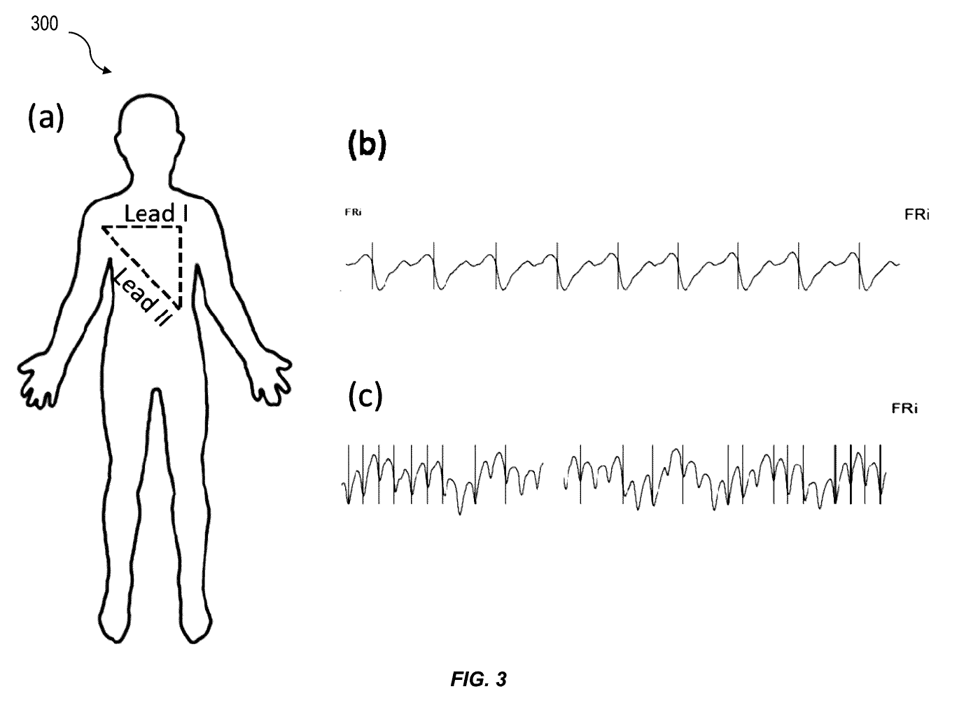

FIG. 3 is a series of plots 300 illustrating: (a) measurement leads used in impedance respiration monitoring; (b) a typical, clean impedance respiration waveform with a rate of 16 breaths per minute; and (c) an impedance respiration waveform corrupted by the cardiac artifact. The actual respiration signal can be seen in the baseline shift. The monitoring algorithm falsely detects the cardiac artifact as breaths and displays a false high respiration rate.

Patient data collected from patient monitors confirmed that, as expected, the cardiac artifact is approximately periodic in short time windows, with a period that coincides with that of the HR as computed from the ECG. In practice, the cardiac artifact, like the ECG, exhibits small frequency variations as a function of the respiration cycle due to Sinus Arrhythmia, and its morphology varies slowly with time, but for the purposes of this work it can be approximated as periodic within small time windows (i.e. from cycle to cycle). Lastly, it was assumed that the cardiac artifact is additive to the respiration waveform.

Using these observations, an adaptive filter was designed in order to reject the cardiac artifact by subtracting a previous sample of the respiration signal from the current sample, using a delay equal to the period corresponding to the HR as computed from the ECG. The filtering equation is given by:

\tilde{r}[n]=\hat{r}[n]−\hat{r}[n−N_{HR}]

where \hat{r}[n] is the current respiration sample and \hat{r}[n−N_{HR}] is an older sample that occurred N_{HR} samples in the past, where N_{HR} is the period of the ECG. The theoretical foundation of the filter follows.

Assuming a noise-free environment the sensed respiration signal \hat{r}[n] is, as mentioned above, the addition of two signals: the true respiration signal, r[n] and the cardiac artifact c[n]:

\hat{r}[n]=r[n]+c[n] (1)

Assuming that c[n] is a periodic signal with period N_{HR}:

c[n]=c[n−N_{HR}] (2)

Where the period N_{HR} of the cardiac artifact can be recovered in real-time using the ECG signal. Introduced is a filtered respiration signal \tilde{r}[n], where the filtered signal is the difference of a previous respiration sample (delayed from the current sample) from the current respiration sample, where the delay of the previous sample is equal to the ECG period N_{HR} as extracted from the ECG:

\tilde{r}[n]=\hat{r}[n]−\hat{r}[n−N_{HR}] (3)

Substituting (1) into (3) yields:

\tilde{r}[n]=r[n]+c[n]−r[n−N_{HR}]−c[n−N_{HR}] (4)

And by inserting (2) into (4):

\tilde{r}[n]=r[n]−r[n−N_{HR}] (5)

Thus, the filtered signal is equivalent to the difference between the current true respiration sample minus another true respiration sample which occurred NHR samples before.

The filter of eq. (3) assumes that the amplitude of the cardiac artifact is time-invariant. However, this is not always the case; data has shown that its amplitude is tied to the amplitude of the R-wave of the ECG, as would be expected. The R-wave amplitude is modulated as a function of the respiration cycle. To compensate for this time-dependence, introduced is an adaptive weight w to the filtering operation:

\tilde{r}[n]=\hat{r}[n]−w·\hat{r}[n−N_{HR}] (6)

where w is a normalizing factor taking values between 0 and 1, and its value is a function of the value of the current R-wave as it is read from the ECG (i.e., the current R-wave as opposed to earlier sensed R-waves). Before determining the weight w, the algorithm determines a time interval, which for example can be equal to one breathing cycle. Then, for each breathing cycle, the amplitude of the R-waves falling in that cycle can be computed.

As an example, assume that two R-waves, R₁ and R₂ occur in a breathing cycle, with corresponding amplitudes A₁ and A₂, where A₂ is the maximum. The weight w₁ corresponding to R₁ will be equal to A₁/A₂, while the weight w₂ corresponding to R₂ will be 1. The algorithm then computes the interval N_{HR} as above. Then, N_{HR} is centered on each R-wave, the same interval is identified in the respiration waveform, and the weight w corresponding to that interval multiplies all samples in the interval.

There are several possible modifications of the suggested filter. One is a generalization where from the current sample a weighted sum of a multitude of past samples instead of just one is subtracted. This is possible if we assume that the cardiac artifact does not change much in morphology in short time intervals, for example within one or two breathing cycles. Under this assumption, eq. (5) can be generalized as:

$$\tilde{r} [ n ] = r [ n ] - \frac{1}{M} {\sum }_{k = 1}^{M} r [ n - k \cdot {N}_{HR} ] \qquad ( 7 )$$

where M is the number of samples during which it is assumed that the cardiac artifact morphology is time-invariant. In the same fashion, eq. (6) can be generalized as:

$$\tilde{r} [ n ] = r [ n ] - {\sum }_{k = 1}^{M} {w}_{k} \cdot r [ n - k \cdot {N}_{HR} ] \qquad ( 8 )$$

where wₖ is a weight corresponding to a respective R-wave interval, and which can be computed as described above. Alternatively, the weights of eq. (8) can be set to take smaller values for larger values of k, and larger values for smaller values of k, to account for the fact that further back in time the cardiac artifact is more likely to be different, and thus should be taken less into account compared to samples closer to the current time-point. Note that if all weights are set to 1/M, then eq. (8) becomes equivalent to eq. (7). In some variations (including those where all weights are set to 1/M), the sum of weights is not greater than 1, so as not to weigh previous samples more than the current sample. Generalizations such as eq. (7), (8) are useful because the averaging operation involved allows for filtering of the noise, and thus for more accurate filtering results.

Another way to improve the accuracy of the filter, is to modify eq. (5) as:

\tilde{r}[n]=r[n]−0.5·(r[n−N_{HR}]+r[n−N_{HR}−1]) (9)

or, as

\tilde{r}[n]=r[n]−0.5·(r[n−N_{HR}]+r[n−N_{HR}+1]) (10)

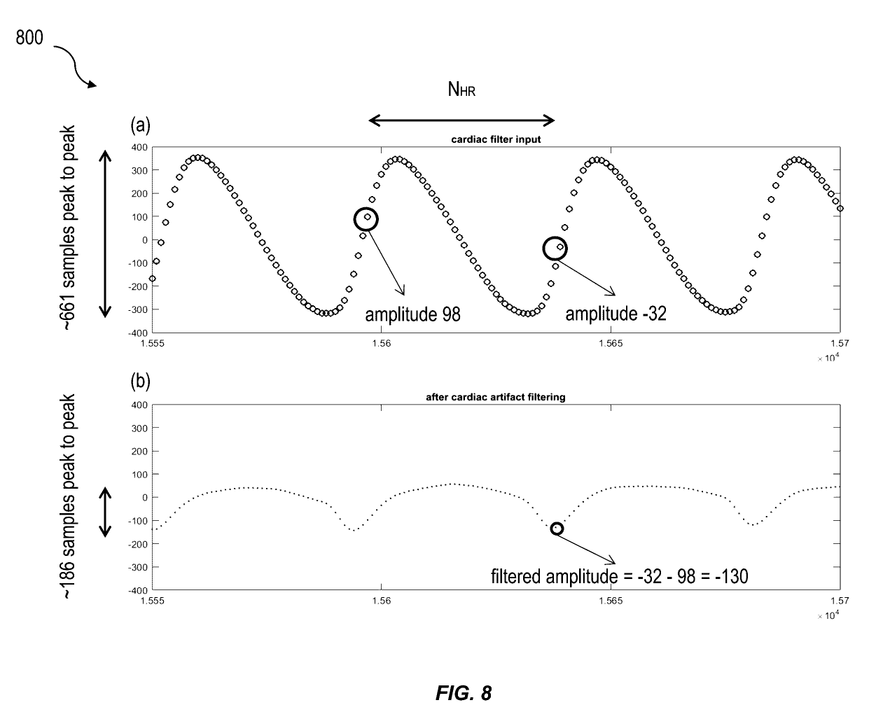

The modifications of eq. (9), (10) can become especially relevant when there are fast variations in the respiration waveform which are sparsely sampled, because in such cases eq. (5) or (6) can be corrupted by quantitation errors. As an example, consider FIG. 8. In FIG. 8 (a), an apnea event is shown where the only signal present is the cardiac artifact. This waveform has been sampled using a sampling rate of 50 samples/sec, and the heart rate is equal to 70 bpm. When converting the heart rate to a sample interval, we get

$${N}_{HR} = \frac{{F}_{s} \cdot 60}{HR} = \frac{3000}{70} = 42.85$$

samples, which would be rounded to 42 samples for a fixed-integer processor. This rounding error, in addition to the fact that the sharp rises are sparsely sampled, leads to a filtered waveform produced by eq. (5) that is smoother than the raw waveform, but not flat enough for the artifact not to be detected as breaths. Hence, this effect can lead to missing an apnea. Even if the rounding error is corrected for, there are still scenarios where the filtered waveform would not be completely flat. For example, for a heart rate of 80 bpm,

$${N}_{HR} = \frac{{F}_{s} \cdot 60}{HR} = \frac{3000}{80} = 37.5 ,$$

in which case there is a significant rounding error whether we round N_{HR} to 37 or 38. In such cases, the use of eq. (9) or (10) can compensate for the rounding error and the sparse sampling of the waveform by taking the average of two neighboring samples (in effect, by interpolating). Eq. (9) should be used when rounding N_{HR} up, whereas eq. (10) should be used when rounding down, as an example in cases where before rounding, N_{HR} takes values ending in decimals between 0.4 and 0.6.

During real-time monitoring, there are time instances when filtering may be unnecessary. In particular, there are instances when the cardiac artifact is sufficiently weak not to significantly distort the respiration waveform, and filtering a clean respiration signal could in turn introduce distortions. However, it is possible to turn off the filter operation if the signal power to cardiac artifact power ratio (SCR) is greater than a preset threshold. The power of the cardiac artifact can be estimated by integrating the power density of the respiration waveform across a frequency region centered on the heart rate as computed by the ECG. The power of the respiration rate can be estimated in the same way by integrating the power density across the other largest peak of the power spectrum (which falls within clinically feasible respiration rates), and then the SCR can be computed. When the SCR is below a threshold, then the adaptive filter can be activated. In addition, during ventricular arrhythmias, such as ventricular fibrillation episodes, the ECG signal becomes chaotic, and it may not be possible to use the ECG rate effectively to reject the cardiac artifact. However, ventricular arrhythmias are critical events, which put a patient's life at risk, and patient monitors issue high priority alarms during such events. Thus, during ventricular arrhythmias, the accuracy of the respiration rate becomes unimportant, and the filtering operation should be deactivated when the ECG is not normal.

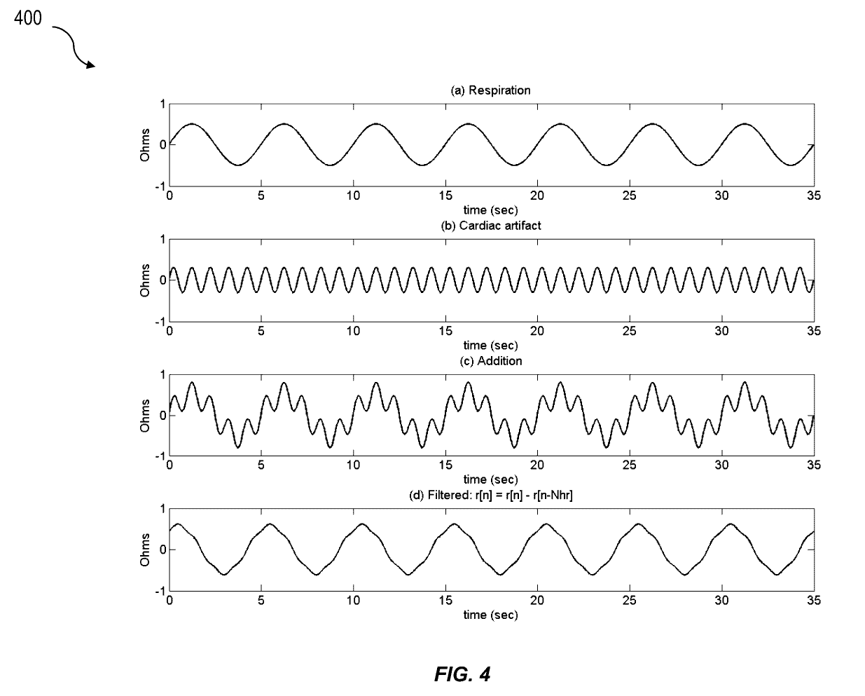

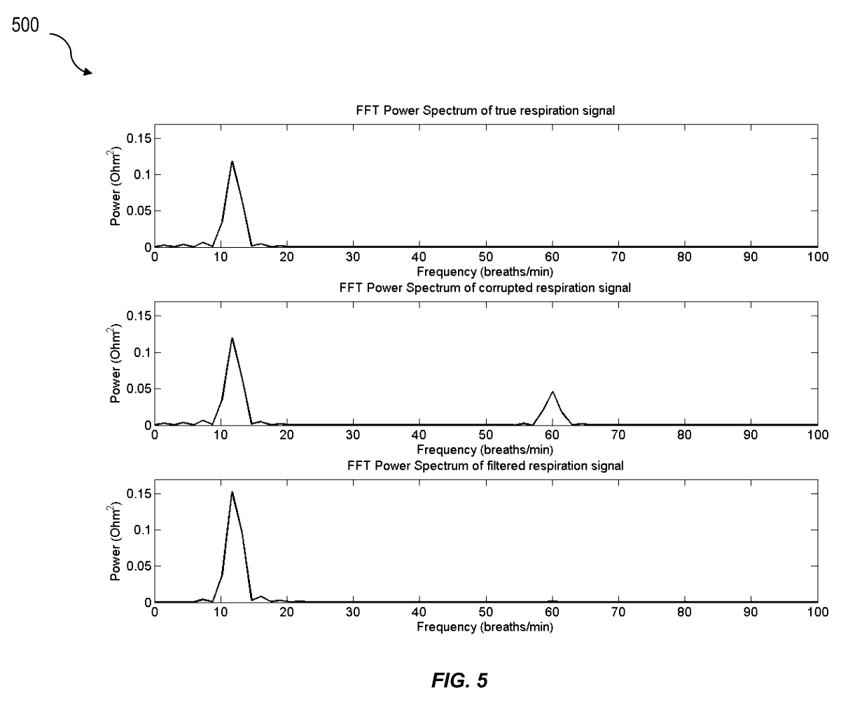

As a proof of concept, the filter was first applied to simulated data. In particular, a clean respiration waveform was simulated as a sinus of 1 Ohm peak to peak amplitude and 12 brpm rate, and then corrupted by an additive cardiac artifact simulated as a sinusoidal waveform of 0.5 Ohm peak to peak amplitude and 60 bpm rate (FIG. 4). For testing purposes, the known cardiac heart rate was given as an input to the filter. As it can be seen from FIG. 4, the adaptive filter eliminated the cardiac artifact almost entirely. In addition, the filtering operation increased the amplitude of the respiration waveform, which improves the effectiveness of a peak detection algorithm used to compute the RR. To quantify the improvement introduced by the adaptive filter, the power spectrum of the signal was computed before and after filtering (FIG. 5). The power spectrum analysis showed that the filter increased the SCR by 17.8 dB.

FIG. 4 is a series of plots 400 illustrating: (a) simulated respiration signal with RR=12 bpm; (b) simulated cardiac artifact with HR=60 bpm; (c) the addition of the simulated respiration and cardiac signals produces the corrupted respiration signal; and (d) the filtered signal is almost clean of the cardiac artifact. In addition, peaks and valleys are emphasized, making the detection of the respiration rate from the filtered signal simpler.

FIG. 5 is a series of plots 500 illustrating: (a) the power spectrum of the simulated true respiration signal of FIG. 2(a); (b) The power spectrum of the simulated corrupted signal of FIG. 4(c): the frequency components of both the true respiration signal and the cardiac artifact can be seen; (c) the power spectrum of the filtered signal of FIG. 4(d): the cardiac artifact component has been removed

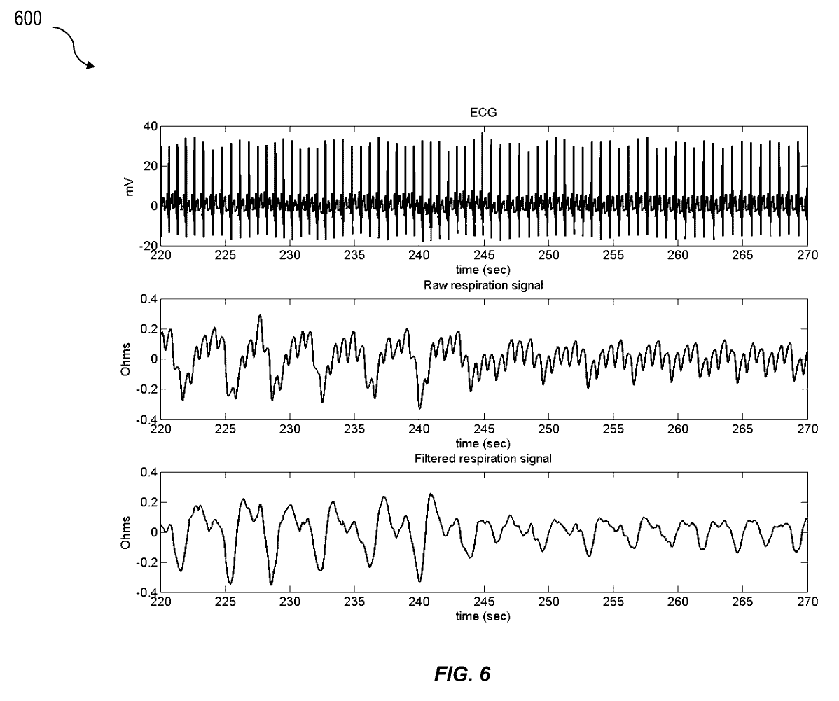

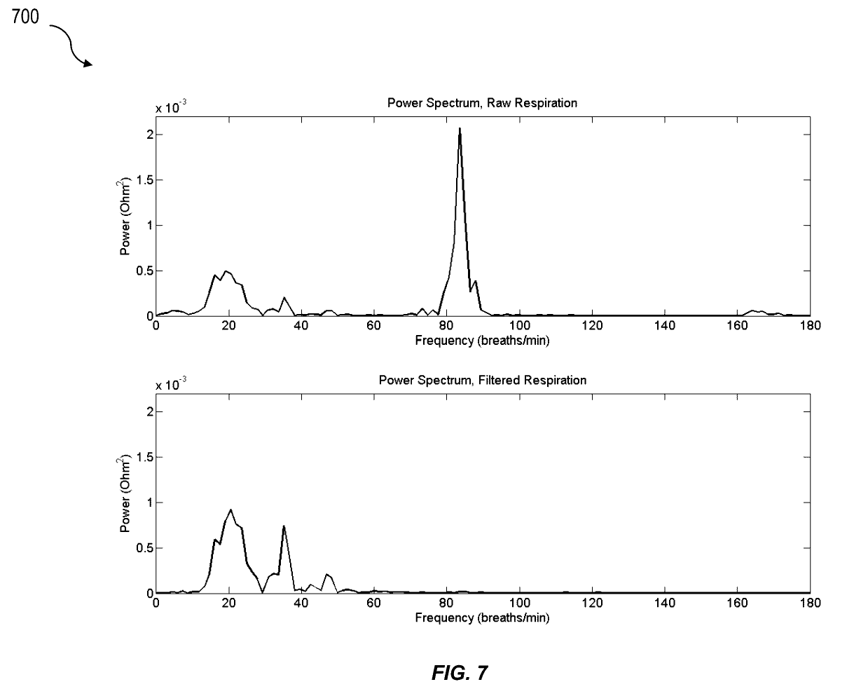

In addition to applying the filter to simulated data, the filter was also applied to data from a human subject. In particular, ECG and IR data from Lead I were collected from a human subject using a patient monitor. Then the adaptive filter was applied offline to the collected data and it successfully rejected the cardiac artifact (FIG. 6). The instantaneous heart rate as calculated from the ECG was input to the filter. In FIG. 7, the power spectrum of the raw and the filtered respiration signal can be seen for the time window 245-265 sec of FIG. 6, where the subject is experiencing shallow breathing. In this case, the filter improved the SCR by 23.5 dB. In the time window 220-240 sec of FIG. 4, the filter improved the SCR by 20 dB (spectrum not shown). Note that in FIG. 7 there are two dominant peaks for the respiration rate. This is because the spectrum is computed for a 20 sec window, during which the respiration rate of the subject increased from 20 brpm to 35 brpm.

FIG. 6 is a series of plots 600 illustrating: (a) ECG signal (50 seconds) used as the input of the adaptive filter; (b) respiration signal (Lead I) before adaptive filtering in which the cardiac artifact is evident; and (c) respiration signal after filtering. The cardiac artifact has been almost completely eliminated.

FIG. 7 is a series of plots 700 illustrating: (a) power spectrum of the raw respiration signal (245-265 sec of FIG. 4b). The cardiac artifact component is dominant; and (b) power spectrum of the filtered respiration signal (245-265 sec of FIG. 4c). The cardiac artifact component has been eliminated.

An adaptive filter has been designed and developed for the rejection of the cardiac artifact in impedance respiration. The cardiac artifact is one of the major causes of false alarms in the hospital setting, as it often causes patient monitors to detect false high respiration rates. The filter was tested against both simulated data, and data from human subjects collected from a patient monitor. In both cases, the filter successfully rejected the cardiac artifact and recovered the underlying respiration waveform, which should then enable the respiration rate calculation algorithm to correctly detect the respiration rate.

As the cardiac artifact period is equal to the heart rate period N_{HR} as extracted from the ECG, which is always significantly smaller than the respiration rate period, the filtering operation will result either in a slightly amplified or attenuated version of the true respiration signal, while at the same time removing the cardiac artifact and increasing the SCR ratio.

In addition, initial real-time trials on reproduced patient respiration waveforms not shown herein demonstrated that the filter successfully prevents the monitor from detecting false respiration rates and from issuing false alarms caused by the cardiac artifact.

The initial results demonstrate that the adaptive filter developed has the potential to substantially reduce false respiration rates, which produce false alarms caused by the cardiac artifact in the hospital setting. In addition, the filter is expected to decrease the occurrence of missed apneas due to the cardiac artifact. In order to quantify this improvement, future work includes the application of the filter in real-time monitoring on human subjects also monitored with an alternative respiration rate detection modality such as etCO2, which will be used as the gold standard. Such a study will allow for quantification of the effective improvement of the filter on respiration rate calculations and on the reduction of false alarms.

Although a few variations have been described in detail above, other modifications or additions are possible. For example, the current subject matter can be implemented with a sensor system that is not a patient monitor but provides impedance respiration data, the current subject matter allowing for adaptive removal of the cardiac artifact in the impedance respiration.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A method for removing cardiac artifacts from respiration waveforms comprising:
receiving a stream of respiration samples of a sensed respiration signal that collectively characterize respiration data for a patient;
receiving heart rate data specifying a heart rate for the patient that is measured concurrently with the sensed respiration signal;
continuously adaptively filtering each current respiration sample in the stream to result in a corresponding filtered respiration signal that removes cardiac artifacts, the filtering subtracting an earlier respiration sample having a delay equal to a period corresponding to the heart rate of the patient from the then current respiration sample; and
promoting the filtered respiration signals.

2. The method of claim 1, wherein promoting the filtered respiration signals comprises at least one of: displaying the filtered respiration signals in an electronic visual display, persisting the filtered respiration signals in a physical data storage device, transmitting the filtered respiration signals over a wired or wireless computing network to a remote computing device, or loading the filtered respiration signals into memory of a computing device.

3. The method of claim 1, wherein the respiration signal comprises an impedance respiration waveform.

4. The method of claim 1, wherein the heart rate data is derived from an electrocardiogram (ECG) electrode set affixed to the patient, and the method further comprises:
extracting the period from the heart rate data.

5. The method of claim 4, wherein the adaptive filtering further comprises:
weighting the earlier respiration samples by a normalizing factor that is based on a value of a corresponding R-wave read from the heart data at a time matching the corresponding current respiration sample.

6. The method of claim 5, wherein the normalization factor is equal to an amplitude of the corresponding R-wave normalized by a maximum R-wave amplitude over a respiration rate period.

7. The method of claim 1 further comprising:
estimating power of the cardiac artifact by integrating a power density of the respiration sensed respiration signal across a frequency region centered on the heart rate.

8. The method of claim 7 further comprising:
estimating power of the respiration rate by integrating a power density across a largest peak of a spectrum corresponding to a respiration rate.

9. The method of claim 8 further comprising:
computing a signal power to cardiac artifact power ratio (SCR) by dividing the estimated power of the respiration rate by the estimated power of the cardiac artifact.

10. The method of claim 9 further comprising:
activating the adaptive filtering when the SCR is below a pre-defined threshold.

11. The method of claim 9 further comprising:
deactivating the adaptive filtering when the SCR is above a pre-defined threshold.

12. The method of claim 1 further comprising:
deactivating the adaptive filtering when the heart rate data indicates a ventricular arrhythmia.

13. The method of claim 1, wherein each of the receiving, receiving, filtering, and promoting are implemented by at least one programmable data processor forming part of at least one computing device.

14. A system comprising:
at least one programmable data processor; and
memory storing instructions, which when executed by the at least one programmable data processor, implement operations comprising:
receiving a stream of respiration samples of a sensed respiration signal that collectively characterize respiration data for a patient;
receiving heart rate data specifying a heart rate for the patient that is measured concurrently with the sensed respiration signal;
continuously adaptively filtering each current respiration sample in the stream to result in a corresponding filtered respiration signal that removes cardiac artifacts, the filtering subtracting an earlier respiration sample having a delay equal to a period corresponding to the heart rate of the patient from the then current respiration sample; and
promoting the filtered respiration signals.

15. The system of claim 14 further comprising an electronic visual display for displaying at least a portion of the promoted filtered respiration signals.

16. The system of claim 15, wherein the at least one programmable data processor, memory, and the display forming part of a patient monitor.

17. The system of claim 13 further comprising:
an electrocardiogram (ECG) circuit; and
electrodes configured to be coupled to the ECG circuit and for affixation to the patient;
wherein the electrodes and the ECG circuit in combination generate the sensed respiration signal.

18. The system of claim 14, wherein the operations further comprise:
estimating power of the cardiac artifact by integrating a power density of the respiration sensed respiration signal across a frequency region centered on the heart rate;
estimating power of the respiration rate by integrating a power density across a largest peak of a spectrum corresponding to a respiration rate;
computing a signal power to cardiac artifact power ratio (SCR) by dividing the estimated power of the respiration rate by the estimated power of the cardiac artifact;
activating the adaptive filtering when the SCR is below a pre-defined threshold; and
deactivating the adaptive filtering when the SCR is above a pre-defined threshold.

19. A non-transitory computer program product storing instructions which, when executed by at least one data processor forming part of at least one computing device, execute operations for removing cardiac artifacts from respiration waveforms comprising:
receiving a stream of respiration samples of a sensed respiration signal that collectively characterize respiration data for a patient;
receiving heart rate data specifying a heart rate for the patient that is measured concurrently with the sensed respiration signal;
continuously adaptively filtering each current respiration sample in the stream to result in a corresponding filtered respiration signal that removes cardiac artifacts, the filtering subtracting an earlier respiration sample having a delay equal to a period corresponding to the heart rate of the patient from the then current respiration sample; and
promoting the filtered respiration signals.

20. A method for removing cardiac artifacts from respiration waveforms, the method being implemented by one or more programmable data processors forming part of at least one computing device and comprising:
receiving, by at least one programmable data processor, a stream of respiration samples of a sensed respiration signal that collectively characterize respiration data for a patient;
receiving, by at least one programmable data processor, heart rate data specifying a heart rate for the patient that is measured concurrently with the sensed respiration signal;
adaptively filtering, by at least one programmable data processor, each current respiration sample in the stream to result in a corresponding filtered respiration signal that removes cardiac artifacts, the filtering subtracting a weighted sum of a plurality of earlier respiration samples from the then current respiration sample; and
promoting, by at least one programmable data processor, the filtered respiration signals.

21. The method of claim 21, wherein each of the plurality of earlier respiration samples has a delay equal to an integer multiple of a period corresponding to the heart rate of the patient.
